(19) United States
(12) Patent Application Publication
(10) Pub. No.: US 2011/0237957 A1
(43) Pub. Date: Sep. 29, 2011

(54) DEVICE, SYSTEM, AND METHOD FOR COMBINED OPTICAL AND THERMOGRAPHIC DETECTION OF THE CONDITION OFJOINTS

(75) Inventor: Wouter H.J. Rensen, Eindhoven (NL)

(73) Assignee: KONINKLIJKE PHILIPS ELECTRONICS N.V., EINDHOVEN (NL)

(21) Appl. No.: 13/133,009

(22) PCT Filed: Nov. 27, 2009
(86) PCT No.: PCT/IB09/55384

§ 371 Date: Jun. 6, 2011

(30) Foreign Application Priority Data
Dec. 5, 2008 (EP) 08170820.8

(51) Int. Cl. A61B 6/00

(52) U.S. Cl. 600/474

(57) ABSTRACT

The invention relates to a device, system and method for optical detection of the condition of a joint. The invention involves: an optical measurement unit (4, 5) for irradiating a subject's body part comprising at least one joint with light and locally detecting attenuation of the light at the at least one joint and at least one other portion of the body part; a thermographic measurement unit (3) for thermographically imaging the subject's body part comprising the at least one joint; a control unit (9) for controlling the device such that data obtained by one of the optical measurement unit (4, 5) and the thermographic measurement unit (3) is used to calibrate data obtained by the other of the optical measurement unit (4, 5) and the thermographic measurement unit (3).

### FIELD OF INVENTION

The present invention relates to an optical detection method and to a device for optical detection of the condition of joints.

### BACKGROUND OF THE INVENTION

In the context of the present application, the term light is to be understood to mean non-ionizing electromagnetic radiation, in particular with wavelengths in the range between 400 nm and 1400 nm. The term body part means a part of a human or animal body. The term blocking covers both complete blocking and blocking to a substantial extent.

In general, the present invention relates to optical detection of joint conditions, in particular to the optical detection of joint diseases such as rheumatoid arthritis (RA). The treatment of such joint diseases is staged. Usually, a patient first receives pain killers. These are frequently followed by non-steroid anti-inflammatory drugs (NSAIDs) and disease modifying anti-rheumatic drugs (DMARDs). In many cases, the last stage in treatment with drugs is the use of biological therapies. In particular the last category is expensive and treatment can cost tens of thousands of dollars per year per patient. Additionally, the drugs used in later stages of treatment often cause more severe side effects. With respect to such joint diseases, medical professionals base their decisions on changes in therapy on disease activity which is given by the number and the severity of inflamed joints.

Since rheumatoid arthritis is a progressive disease and early diagnosis and start of treatment can help postponing adverse effects and high costs of treatment, there is a demand for methods and devices for providing satisfactory information about the condition of joints and which assist a medical professional to come to a conclusion with respect to the actual joint condition.

It has been found in time-dependent measurements using non-targeted fluorescent dyes administered to the patient that perfusion dynamics in diseased joints are different as compared to normal healthy joints. However, in the clinical practice of rheumatologists, administration of contrast agents is impractical in most cases.

As an alternative, it has been proposed to use Diffuse Optical Tomography (DOT) to image joints for providing information about their condition. In a research project, venous blood flow to a body part has been temporarily obstructed by means of a pressure cuff and a single joint has been imaged by means of DOT. In such studies, it has been found that optical parameters exist which correlate with the presence of rheumatoid arthritis (RA).

For example, it is known that inflammation can be recognized by a change in perfusion. Blood constituents, in particular both oxygenated and deoxygenated hemoglobin have distinct optical characteristics compared to other constituents of the human or animal body and thus can in principle be optically detected.

A device and method for detecting the condition of a joint is described in European patent application EP08156917.0. The document describes measuring the inflammation of a joint by analyzing changes in spectral transmission of joints and other parts of, for instance, a patient's hand before, during, and after (partial) occlusion of blood flow in the patient's arm and hand. Optical detection methods are especially suited for studying smaller joints such as the joints in a human finger or human toe. When imaging larger joints, for instance, elbows, shoulders, hips, knees, or ankles, light transmission through such joints becomes increasingly difficult with increasing size of the joints. It has been attempted to use thermographic methods to establish disease activity in larger joints like elbows, knees, and ankles (see, for instance, R. Salisbury et al., “Heat Distribution over Normal and Abnormal Joints-Thermal Pattern and Quantification”, Annals of the Rheumatic Diseases 42(5), 494-499 (1983); L. Jiang et al., “A Perspective on Medical Infrared Imaging”, Journal of Medical Engineering & Technology 29(6), 257-267 (2005); B. Sanchez et al., “Use of a Portable Thermal Imaging Unit As a Rapid, Quantitative Method of Evaluating Inflammation and Experimental Arthritis”, Journal of Pharmacological and Toxicological Methods 57(3), 169-175 (2008)). However, these methods have never been widely adopted. One reason for this is that these methods require a patient to be in a room that is free of draft and thermally stable within 1° C. for at least 15 minutes.

### SUMMARY OF THE INVENTION

It is an object of the invention to provide a device and method for detecting the condition of a joint which enables obtaining enhanced data from both smaller and larger joints.

The object is solved by a device according to claim 1. The invention is based on the recognition that using data obtained by one measurement unit can be used as a baseline to calibrate data obtained by the other measurement unit. For instance, data from the optical measurement unit can be enhanced by data from the thermographic measurement unit. In this way, obtaining attenuation data from, for instance, smaller joints benefits from data obtained by the thermographic measurement unit. It should be noted that a wrist is a medium-sized joint the condition of which may be assessed by both optical and thermographic methods.

An embodiment of a device according to the invention is characterized in that the control unit is arranged for calibrating data obtained by the thermographic measurement unit on the basis of data obtained by the optical measurement unit. This embodiment has the advantage that it allows obtaining data on smaller joints using the optical measurement unit as well as obtaining data on larger joints using the thermographic measurement unit, but without the aforementioned constraints relating to thermal stability. By obtaining optical data as well as thermographic data from a smaller joint, data from the thermographic measurement unit can be calibrated allowing subsequent thermographic measurements of a larger joint that is not yet in a thermal equilibrium with its environment.

The object of the invention is also realized with a system for optical detection of the condition of a joint according to claim 3. Having a thermal modulator in addition to a device according to any one of the previous embodiments has the advantage that it enables modulating the temperature of the subject's body part. This in turn enables studying the response of the body part to changes in temperature, changes being potentially different for diseased and healthy tissues. Moreover, applying thermal modulation further reduces the need for thermal stabilization. Not the absolute temperature of the body part under investigation is a measure for disease activity, but its thermal response to the thermal modulation that is applied.

An embodiment of the system according to the invention is characterized in that the thermal modulator comprises at least one element chosen from the group comprising: a bloodflow blocking unit, a blower, a radiator, an evaporation medium. This embodiment has the advantage that a bloodflow blocking unit (for instance a pressure cuff), a blower (for instance a fan for blowing hot and/or cool air), a radiator (for instance an infrared heat radiator), and an evaporation medium (for instance a fluid like ethanol on the skin of a subject's body part that is investigated) provide easy means for thermally modulating a subject's body part. In general any method or device for applying a thermal modulation to a subject's body part can be used.

The object of the invention is also realized with a detection method according to claim 6. The method achieves the aforementioned benefits.

An embodiment of a method according to the invention is characterized in that in the step of calibrating data obtained in the step of optically imaging a subject's body part is calibrated using data obtained in the step of thermographically imaging the body part. This embodiment has the advantage that it allows obtaining data on smaller joints using the optical measurement unit as well as obtaining data on larger joints using the thermographic measurement unit, but without the aforementioned constraints relating to thermal stability. This benefit was already discussed in relation to an embodiment of the device according to the invention.

A further embodiment of a method according to the invention is characterized in that data of distinct local attenuation measurements and thermographically acquired data which are acquired substantially simultaneously for the at least one joint and for the at least one other portion of the body part are compared to each other. This embodiment has the advantage that valuable information for judging the condition of the at least one joint can be obtained by comparing data obtained for the joint with data obtained substantially simultaneously from the at least one other portion of the body part. Comparing data that has been obtained substantially simultaneously as the advantage that changes affecting the at least one joint and the at least one other portion simultaneously are cancelled, leaving only information relating to time-independent differences between the at least one joint and the at least one other portion.

A further embodiment of a method according to the invention is characterized in that the at least one other portion of the body part is another joint. This embodiment has the advantage that the response of different joints to changes in blood flow can be compared and information about differences in the condition of several joints is provided. In a preferred embodiment, all joints in both hands of a subject are measured simultaneously.

### BRIEF DESCRIPTION OF THE DRAWINGS

FIG. 1 schematically shows an embodiment of a device according to the invention;

FIG. 2 schematically shows an optical measurement unit for use in a device according to the invention;

FIG. 3 schematically shows an embodiment of a system according to the invention;

FIG. 4 schematically shows an embodiment of a method according to the invention.

### DETAILED DESCRIPTION OF EMBODIMENTS

FIG. 1 schematically shows an embodiment of a device according to the invention. In the illustration, a human body 6 is schematically shown as a body and a hand forms the body part to be examined. However, it should be noted that the invention is not restricted to human bodies and e.g. animal bodies may be subjected to examination. Further, the body part is not restricted to a hand but may also be formed by another body part comprising at least one joint such as arms, legs, feet, etc.

In the embodiment shown, the device for optical detection of the condition of joints comprises a thermographic measurement unit 3, an optical measurement unit 4, 5, a blood flow blocking unit 8, and a control unit 9. The control unit 9 is provided to control the operation of the device and data acquisition. The optical measurement unit 4, 5 is provided to irradiate portions of the body part under examination with light and measure the local attenuation of the light at different positions of the body part. For example, in the embodiment shown the optical measurement unit 4, 5 is formed by a measurement head which will be described in more detail below. The blood flow blocking unit 8 is provided for temporarily blocking the blood flow to and/or from the body part under examination. In the embodiment, the blood flow blocking unit 8 is provided by a pressure cuff surrounding the arm to which the hand under examination belongs and obstructing the blood flow by application of pressure to the upper arm. It should be noted that the blood flow blocking unit 8 may be adapted differently in order to allow temporarily at least partially blocking the blood flow to and/or from body parts other than a hand. The thermographic measurement unit 3 is used to obtain thermographic data from the body part under investigation. According to an embodiment of the device according to the invention, data obtained by the optical measurement unit 4, 5 is used to calibrate data obtained by the thermographic measurement unit 3 enabling subsequent thermographic measurements of larger joints, for instance, wrists, elbows, shoulders, hips, knees, and ankles without stringent thermal stability requirements. In the prior art, for instance, thermographic measurements require thermal stability within 1° C. for 15 minutes. The thermographic measurement unit 3 applies methods that are well known in the prior art such as an InGaAs camera, or any other method to provide a thermal image of the body part under investigation. Preferably, the thermographic measurement unit 3 and the subject are positioned relative to each other such that the thermographic measurement unit 3 has all relevant larger joints in its field of view. In this way, it is not necessary to take separate thermographic images from different joints of the subject or to have multiple thermographic measurement units.

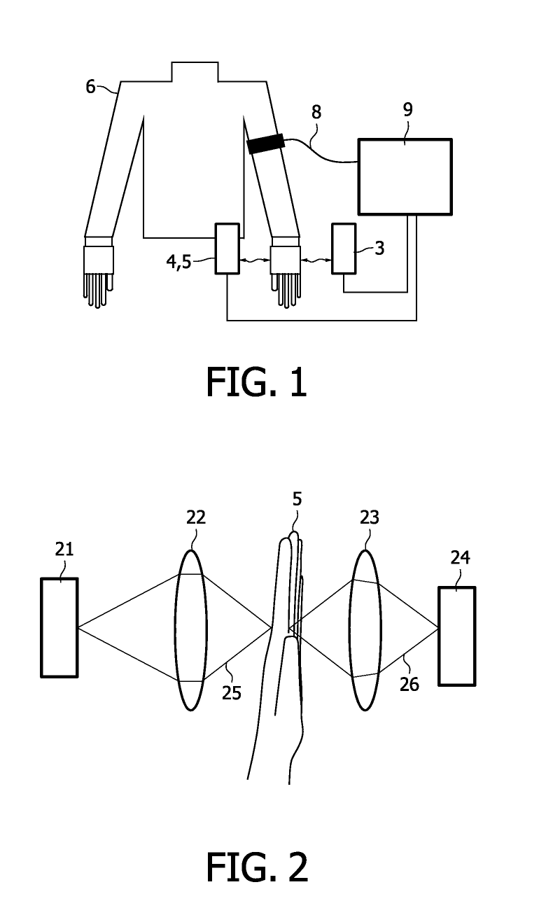

The construction of the optical measurement unit 4, 5 according to the embodiment will be described in further detail with reference to FIG. 2.

The optical measurement unit 4, 5 schematically shown in FIG. 2 is adapted for attenuation measurements in transmission geometry. The optical measurement unit 4, 5 comprises a light source unit 21 emitting a beam of light for irradiating the body part. The light source unit 21 comprises at least one light source and appropriate light guides to direct the beam of light to the body part. The light source may be formed by a lamp or by one or more lasers and the light guides may for instance be formed by optical fibers. The light source unit 21 is adapted to be capable to emit light of at least two different wavelengths, preferably in the red to near infrared, wherein one wavelength is chosen such that blood has a high absorption and another wavelength is chosen such that the absorption of blood is low or comparable to surrounding tissue. Suitable wavelengths are for instance 600 nm and 805 nm but other wavelengths fulfilling these criteria are possible as well. Wavelengths in the wavelength range between 550 and 980 nm are particularly suitable. Further, an optical component 22 which e.g. may be formed by a lens is provided for directing the light to the body part. The optical component 22 is capable of concentrating the light (irradiation light 25) on a specific area of interest (or several specific areas of interest; i.e. specific positions) of the body part as will be described below. A second optical element 23 is provided to collect light emerging from the specific area (or areas) of interest and direct the collected light 26 to a detection element 24. The detection element 24 may for instance be formed by a photodiode, a CCD, an optical guide such as a fiber connecting to a photodiode, or another light detection scheme known in the art.

The optical measurement unit 4, 5 is adapted such that distinct local attenuation measurements for at least two different portions of the body part can be performed.

The control unit 9 is adapted such that it controls at least partial blocking of the blood flow to and/or from the body part by means of the blood flow blocking unit 8. Further, it controls the optical measurement unit 4, 5 such that local attenuation measurements are performed before the blood flow is blocked, local attenuation measurements (at the same positions) are performed during the blocking of blood flow, and local attenuation measurements (at the same positions) are performed after restoring the blood flow.

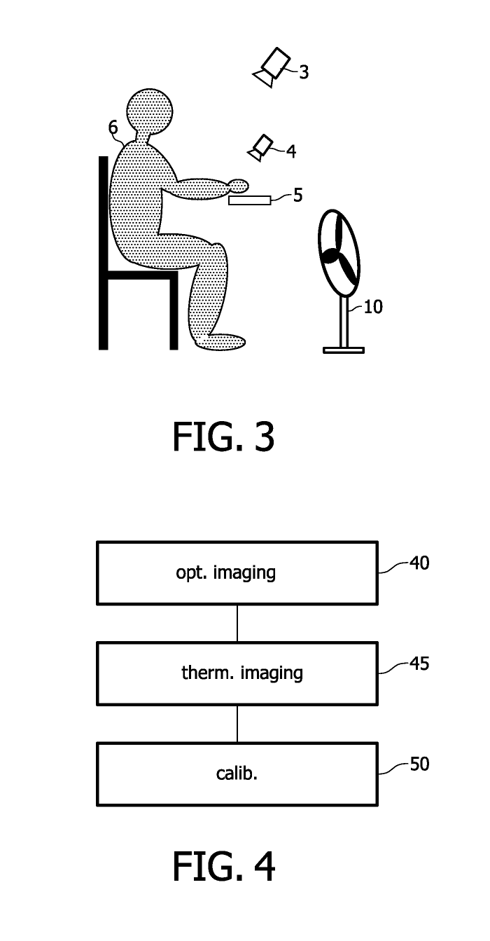

FIG. 3 schematically shows an embodiment of a system according to the invention. The figure shows a subject 6, an optical measurement unit 4, 5, and a thermographic measurement unit 3. This setup is essentially the same as that shown in FIG. 1 to which the reader is referred. In the present figure the subject 6 is sitting down. However, it is also possible to perform measurements with a subject 6 in other positions such as standing up or lying down. Element 5 in the optical measurement unit 4, 5 is a light source for irradiating the body part under investigation. Element 4 in the optical measurement unit 4, 5 is a detector for locally detecting attenuation by the body part under investigation of light generated by the light source. The system shown additionally comprises a thermal modulator 10. In the present embodiment, the thermal modulator 10 is a blower for blowing hot and/or cool air along the body part under investigation. The optical measurement unit 4, 5 and the thermographic measurement unit 3 may be used to study the response of the body part to the thermal modulation applied by the thermal modulator 10. In that case, it is the response to the thermal modulation and not the absolute temperature of the body part that is used to obtain information about the condition of the body part comprising the joint of interest. In general, the thermal modulator 10 may be any device or product suitable to provide thermal modulation. Other examples are: a blood flow blocking unit (for instance a pressure cuff), a radiator (for instance an infrared heat radiator), and an evaporation medium (for instance a fluid like ethanol). Obviously, blocking the blood flow, irradiating with heat, and evaporating a medium from a surface of the body part will modulate the temperature of that body part.

FIG. 4 schematically shows an embodiment of a method according to the invention. In step 40, a subject's body part comprising at least one joint is optically imaged by irradiating the subject's body part with light and detecting local attenuation of the light by the body part at the position of the at least one joint and at the position of at least one other portion of the body part. In step 45 the body part is thermographically imaged. In step 50 data obtained in one of the steps of optically imaging a subject's body part and thermographically imaging the body part is calibrated using data obtained in the other of the steps of optically imaging a subject's body part and thermographically imaging the body part.

It should be noted that the above-mentioned embodiments illustrate rather than limit the invention, and that those skilled in the art will be able to design many alternative embodiments without departing from the scope of the appended claims. In the claims, any reference signs placed between parentheses shall not be construed as limiting the claim. The word “comprising” does not exclude the presence of elements or steps other than those listed in a claim. The word “a” or “an” preceding an element does not exclude the presence of a plurality of such elements. In the system claims enumerating several means, several of these means can be embodied by one and the same item of computer readable software or hardware. The mere fact that certain measures are recited in mutually different dependent claims does not indicate that a combination of these measures cannot be used to advantage.

1. Device for optical detection of the condition of a joint, the device comprising:
an optical measurement unit (4, 5) for irradiating a subject's body part, the body part comprising at least one joint, with light and locally detecting attenuation of the light at the at least one joint and at least one other portion of the body part;
a thermographic measurement unit (3) for thermographically imaging the subject's body part comprising the at least one joint;
a control unit (9) for controlling the device such that data obtained by one of the optical measurement unit (4, 5) and the thermographic measurement unit (3) is used to calibrate data obtained by the other of the optical measurement unit (4, 5) and the thermographic measurement unit (3).

2. Device according to claim 1, wherein the control unit (9) is arranged for calibrating data obtained by the thermographic measurement unit (3) on the basis of data obtained by the optical measurement unit (4, 5).

3. System for optical detection of the condition of a joint, the system comprising:
a device according to claim 1;
a thermal modulator (10) for modulating the temperature of the subject's body part comprising the at least one joint.

4. System according to claim 3, wherein the thermal modulator (10) comprises at least one element chosen from the group comprising: a bloodflow blocking unit, a blower, a radiator, an evaporation medium.

5. Device according to claim 1, wherein the device or system is a medical device or a medical system respectively.

6. Detection method comprising the following steps:
optically imaging (40) a subject's body part comprising at least one joint, the optical imaging comprising the steps of:
irradiating the subject's body part comprising the at least one joint with light;
detecting local attenuation of the light by the body part at the position of the at least one joint and at the position of at least one other portion of the body part;
thermographically imaging (45) the body part;
calibrating data (50) obtained in one of the steps of optically imaging a subject's body part and thermographically imaging the body part using data obtained in the other of the steps of optically imaging a subject's body part and thermographically imaging the body part.

7. Detection method according to claim 6, wherein in the step of calibrating data (50) data obtained in the step of optically imaging a subject's body part is calibrated using data obtained in the step of thermographically imaging the body part.

8. Detection method according to claim 6, wherein the method further comprises the following step:
modulating the temperature of the body part.

9. Detection method according to claim 8, wherein modulating the temperature of the body part is done using at least one procedure chosen from the group comprising: obstructing bloodflow to the body part, blowing a gas along the body part, irradiating the body part with heat radiation, evaporating a medium from a surface of the body part.

10. Detection method according to claim 6, wherein data of distinct local attenuation measurements and thermographically acquired data which are acquired substantially simultaneously for the at least one joint and for the at least one other portion of the body part are compared to each other.

11. Detection method according to claim 6, wherein the at least one other portion of the body part is another joint.
